(19) United States
(12) Patent Application Publication
(10) Pub. No.: US 2006/0120913 A1
(43) Pub. Date: Jun. 8, 2006

(54) Method for deodorising large-scale plants

(75) Inventor: Robert Wuest, Rosheim (FR)

Correspondence Address: FOLEY AND LARDNER LLP;SUITE 500, 3000 K STREET NW, WASHINGTON, DC, 20007, US

(73) Assignee: AIR & D - SARL

(21) Appl. No.: 10/525,375

(22) PCT Filed: Nov. 11, 2003
(86) PCT No.: PCT/EP03/12553

§ 371 Date: Feb. 23, 2005

(30) Foreign Application Priority Data
Nov. 15, 2002 (DE) 102 53 257.5

(51) Int. Cl. A61L 9/04

(52) U.S. Cl. 422/4; 422/5; 239/34

(57) ABSTRACT

The invention relates to a method for deodonising large-scale plants, in which solid or liquid malodorous materials are stored, by means of treatment of the unpurified air above the surface of the malodorous materials with active agents which react with or mask malodorous substances in the air. The active agents are dispersed in a matrix of polymers comprising cross-linked hydrophilic groups and together with the above form a spongy mass from which the active agents are slowly released and vaporised. The spongy material is incorporated between two parallel plates (3), which are mounted above the surface of the malodorous materials or at the edge of the large-sized plant and an airflow runs between the plates (3) and releases the active agents.

The present invention relates to a method for deodorizing large-scale plants, in which solid or liquid malodorous materials are stored open-air, by treatment of the unpurified air above the surface of the malodorous materials with active agents that react with or mask the malodorous substances in the air.

Examples of large-scale plants in which malodorous substances are stored are:
Sedimentation tanks that have a sloping edge and can be several hundred square meters in size. They are generally filled with industrial effluent that contains considerable amounts of solids.
Wastewater treatment plants with round tanks, in which domestic and industrial wastewater as well as wastewater from animal processing facilities is treated.
Composting facilities for domestic and garden waste and for industrial sludge.
Landfills on which domestic and industrial waste is stored.
Waste sorting facilities.
Wastewater stacking tanks, for example in the sugar industry, agricultural industry and chemical industry.
Harbor basins and open canals, particularly wastewater canals.
Open-air stables, particularly pigsties.
Big industrial facilities with various types of emissions.

The air above the surface of such plants is saturated with malodorous gases, such as ammonia, amines and sulfur compounds. In order to deal with the odor problem and the pollution of the environment both in the immediate vicinity and further away, expensive measures have to be used, such as covering, storage in enclosed spaces and installation of chimneys and extraction equipment, but these are generally unsatisfactory.

There is therefore a need for efficient and rapidly acting removal or at least considerable mitigation of this odor nuisance.

The resulting object is solved by the method according to the invention. Accordingly, the object of the invention is a method for deodorizing the abovementioned large-scale plants by treating the unpurified air with active agents that react with or mask the malodorous substances in the air, the active agents being dispersed in a matrix of crosslinked polymers containing hydrophilic groups and together with the above form a spongelike composition from which the active agents are slowly released and vaporize. The method is characterized in that the spongelike composition is incorporated between two parallel plates that are mounted above the surface of the malodorous materials or at the edge of the large-scale plant and a stream of air flows between the parallel plates and over the spongelike composition, releasing the active agents.

WO 01/78794 A2 describes an anhydrous gel element for deodorizing air or enclosed spaces that is produced by crosslinking a functionalized liquid polymer with a crosslinking agent in the presence of the deodorizing agent. The gel element can be attached to lattices that are installed in a housing that is in contact with the surrounding air. There is no mention of there being specific release of the deodorizing agent by means of a stream of air. Therefore, no large-scale plants can be deodorized with such equipment.

Suitable matrix polymers are crosslinked polycondensates containing hydrophilic groups and crosslinked (meth)acrylic polymers.

Preferred are condensation products of a maleinized or epoxidized polymer and a crosslinking agent, preferably a polyamine. Suitable polymers are for example reaction products of polydienes, such as polybutadiene, polydecadiene and soybean oil with maleic anhydride, also copolymers of olefins such as ethylene with maleic anhydride, and epoxidized polybutadiene. Preferred crosslinking agents are polyamines, particularly polyoxypropylenediamine and polyoxypropylenetriamine. Urea, polyethylenimine and triethylene glycol are also suitable as crosslinking agents. The crosslinking reaction can take place in the presence of the active agent and/or in alcoholic solution, for example in dipropylene glycol, at elevated temperature. Particularly effective hydrophilic groups are the —CRH—O—groups originating from polyoxyalkylene polyamines, and also the maleic anhydride and carboxyl groups or epoxide groups or the —NR—CO—groups of the crosslinked polymer.

Another class of crosslinked polymers is copolymers of monofunctional (meth)acrylic monomers, for example hydroxyethyl acrylate or poly(propylene oxide)(ethylene oxide) monomethacrylate, with a polyfunctional (meth)acrylic monomer, for example ethyleneglycol dimethacrylate or polyethyleneglycol-400-dimethacrylate as the crosslinking agent. The crosslinked (meth)acrylic polymers are prepared by radical copolymerization of the monomers.

A typical composition of the starting materials for the production of the preferred spongy material is as follows:

| | |
|---|---|
| Maleinized or epoxidized polymers | 10 to 30 weight % |
| or monofunctional (meth)acrylic monomers: | |
| Crosslinking agent: | 0.2 to 10 weight % |
| Water: | 0.5 to 20 weight % |
| Flame retardant: | 0 to 20 weight % |
| Active agent: | Remainder |

In both cases, the crosslinked polymers are able to absorb liquids and gases, for example the active agents, a spongy structure being formed. The crosslinked polymer has a spatial network with pores, in which the volatile foreign substances can be sucked up and absorbed, with the result that the polymer swells like a sponge. In the swollen state, the three-dimensional network consists of elementary cells that have a mean volume of 1 to 1000 nm³, preferably from 3 to 200 nm³.

According to the invention, the crosslinked polymer is loaded with an active agent and with it forms a spongelike composition. The active agent is released slowly therefrom and can then react with the malodorous substance present in the plant, for example amines, ammoniac and sulfur compounds and reduce or mask the same. The active agents are generally liquid aldehydes, ketones, alcohols or esters, for example vanillin, eugenol, thymol, geraniol, camphor oil, citronellol, linalool, menthol, coumarin, citral, alpha-pinene, neryl acetate, linalyl acetate, butyl hydroxyl toluene, C7 to C12 aldehydes, salicylic benzyl ester and natural oily essences. Beside chemical reactions per se, for example between hydrogen sulfide or ammonia and aldehydes, there may also be bonds through electrostatic or van der Waals forces, which will at least mitigate the odor perceptibility.

The active agents can be added either during the production of the crosslinked polymers by condensation or polymerization or the crosslinked polymer can be impregnated with the active agents and thus swollen. The active agent should be present in the spongelike composition in amounts of 10 to 90% by weight, preferably 40 to 80% by weight.

The spongelike composition can contain, in addition to the polymer matrix and the active agents, further additives, especially water in amounts of at least 0.1% by weight, preferably from 0.5 to 20% by weight and especially from 1 to 8% by weight, and also from 1 to 20% by weight of flame retardant, such as sugar, azodicarbonamide or bromine compounds as well as powders for preventing caking and sublimation assistants. Water supports the formation of pores and channels in the production of the crosslinked polymers and ensures that the active agents penetrate the pores more easily, are held there and diffuse again uniformly.

It is essential that the active agent is adjusted to the crosslinked polymer to be used and the content of water in such a way that it is released slowly and uniformly from the spongelike composition and that it retains its effect for at least three days, preferably at least one week, and especially more than one month. The agent that is released can react with the malodorous substances in the gaseous phase and/or mask the malodor. Furthermore, the spongelike composition is able to absorb gaseous malodorous substances and thus remove them from the air.

The spongelike composition, which contains the active agents, can be used in the form of spheres, shavings or granules. However, it is preferably used in the form of crumbs, boards or strips with a thickness of from 0.2 to 5 cm, especially from 0.5 to 3 cm, that are advantageously placed on lattices or nets. According to the invention, the spongelike composition is placed between parallel boards. The boards may consist of wood, metal or plastic. Their dimensions are preferably 5×5 cm to 100×100 cm; the space between the boards can be between 2 and 20 cm, especially between 5 and 15 cm. The pair of boards is open on all sides so that the stream of air can flow through from all sides. The boards are preferably attached to vertical posts; however, they can also be hung on ropes or simply laid on the ground.

A stream of air flows between these parallel boards and over the spongelike composition, thus releasing the active agents. In most cases of application, natural wind acts as the stream of air, the air probably being compressed between the parallel boards by the resistance of the spongelike composition and the contact between the air and the active agents thus being intensified.

If this is not sufficient, the stream of air can be intensified by a fan or a ventilator. The agents that are released mix with the gases evaporating from the malodorous substances and can react with these or mask them. By intensifying the stream of air, if possible, and by choosing the active agents and by their reacting with the polymers and possibly the water, the amount of agents that are released can be controlled and thus an adequate odor reduction can be achieved over a greater distance between the point of emission and point of immission.

Preferably, the parallel boards between which the spongelike composition is incorporated in nets or lattices are fixed horizontally on vertical posts. These posts can be distributed at the edge of the large-scale plant and/or in it. For example, a number of parallel boards, for instance more than five, especially between ten and two hundred, can be arranged around a large-scale plant or distributed in it. Thus, when a wind blows from one direction (and it does not make any difference from which direction), not only are the malodorous substances driven away in this direction from the surface of the plant but at the same time the stream of air releases the active agents from the spongelike composition, which are driven away in the same direction and react with the malodorous substances or mask them.

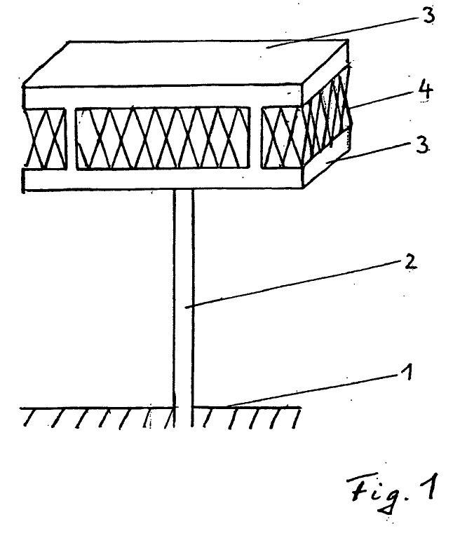

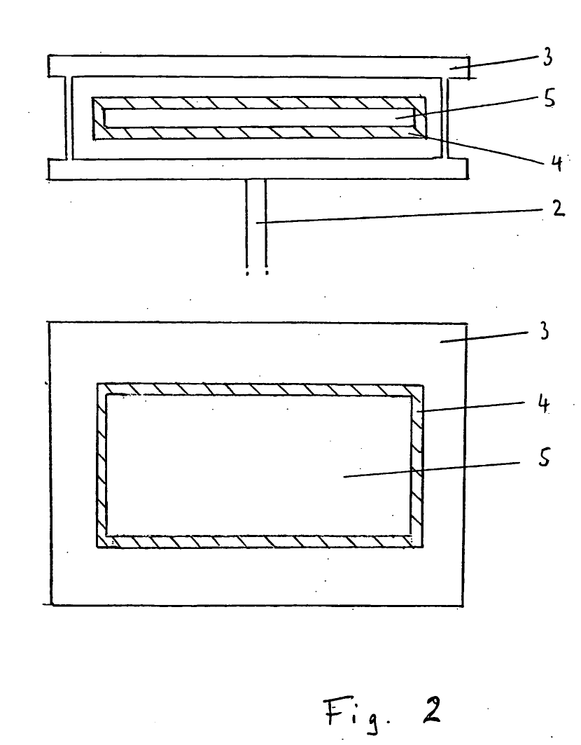

A further subject of the invention is a device for deodorizing large-scale plants that consists of a pair of parallel boards open on all sides between which the spongelike composition is arranged, said composition containing volatile deodorizing agents. There are sketches of such a device in FIGS. 1 and 2. 1 denotes the surface of the malodorous materials, 2 denotes the posts and 3 denotes the parallel boards between which the spongelike composition 5 is arranged in a net or lattice 4.

### EXAMPLE 1

21 g of maleinized polybutadiene (reaction product of liquid polybutadiene with maleic anhydride —LITHENE supplied by Revertex) were mixed with 5 g of water and 79 g of a mixture of oily essences as the active agent at 45° C. (mixture A). 94 g of the active agent and 7.5 g of polyoxypropylene triamine (molecular weight 400) were mixed together (mixture B) Mixtures A and B were stirred together. The spongelike composition that was obtained was cut into boards 2 cm thick, 20 cm wide and 20 cm long and laid on a metal net. This net was arranged between two parallel plastic boards measuring 50×50 cm at a distance of 10 cm. 16 such boards were arranged around the sloping edge of a 100 m² wastewater treatment tank.

### EXAMPLE 2

4 g of poly(propylene oxide)(ethylene oxide) monomethacrylate were mixed with 0.5 g of polyethyleneglycol-400-dimethacrylate in a closed vessel and 100 microliters of 30% hydrogen peroxide were injected. The mixture was evacuated for 30 minutes in an ultrasonic bath. Then the mixture was emptied into an open, shallow vessel and exposed to UV light (290 to 400 nm, 40 mW/cm²) for 5 minutes at 10° C. at a distance of 5 cm.

The crosslinked polymer that was obtained was impregnated with 3 g of salicylic acid benzyl ester as the active agent. The spongelike composition that was obtained was arranged in the device according to the invention. One hundred such devices were attached to posts that were erected over the area of a landfill.

### EXAMPLE 3

15 g of epoxidized polybutadiene (POLY BD supplied by Atofina), 3 g of polyoxypropylene diamine (Jeffamine D 400 supplied by Huntsman) and 8 g of water were mixed with 70 g of a liquid aldehyde, degasified and stirred. The composition was applied to a carrier in a thickness of 5 mm and polymerized by means of a stream of air at 80° C.

Three layers of the sheet that was formed were laid one on top of the other and cut into strips 20 mm wide. These were laid on nets that were incorporated between two boards. Ten such boards were arranged in a composting facility.

1. Method for deodorizing large-scale plants in which malodorous solid or liquid materials are stored open-air by treating the unpurified air above the surface of the malodorous materials with active agents that react with or mask the malodorous substances that the air contains, wherein the active agents are dispersed in a matrix of a crosslinked polymer containing hydrophilic groups and form, with this, a spongelike composition from which the active agents are slowly released and evaporate, characterized in that the spongelike composition is incorporated between two parallel boards that are arranged above the surface of the malodorous materials or at the edge of the large-scale plant and that a stream of air flows between the parallel boards and over the spongelike composition and releases the active agents.

2. Method as claimed in claim 1, characterized in that the spongelike composition in the form of crumbs, boards or strips 0.2 to 5 cm wide is laid on nets or lattices that are incorporated between the parallel boards.

3. Method as claimed in claim 1, characterized in that the crosslinked polymer is a condensation product of a maleinized or epoxidized polymer and a polyamine as the crosslinking agent.

4. Method as claimed in claim 1, characterized in that the crosslinked polymer is a copolymer of a monofunctional (meth)acrylic monomer and a polyfunctional (meth)acrylic monomer as the crosslinking agent.

5. Method as claimed in claim 1, characterized in that the active agents are released slowly and uniformly from the spongelike composition over a period of at least three days.

6. Method as claimed in claim 1, characterized in that the active agents are present in amounts of 10 to 90% by weight in the spongelike composition.

7. Method as claimed in claim 1, characterized in that the active agents are aldehydes, ketones, alcohols, esters or natural oily essences.

8. Method as claimed in claim 1, characterized in that the spongelike composition contains at least 0.1% by weight, preferably 1 to 8% by weight, of water

9. Method as claimed in claim 1, characterized in that the spongelike composition contains additionally flame retardants, sublimation assistants and/or powder in order to prevent caking.

10. Method as claimed in claim 1, characterized in that a number of parallel boards are distributed in the large-scale plant or arranged around its edge.

11. Device for deodorizing large-scale plants consisting of a pair of parallel boards open on all sides between which a spongelike composition is incorporated, said composition containing a matrix of a polymer containing crosslinked hydrophilic agents and volatile deodorizing agents dispersed therein.

12. Device as claimed in claim 11, characterized in that the spongelike composition is laid on nets or lattices.

13. Device as claimed in claim 11, characterized in that the parallel boards measure from 5×5 cm to 100×100 cm and are at a distance of from 2 to 20 cm from each other.

14. Device as claimed in claim 11, characterized in that the parallel boards are fixed horizontally to vertical posts.
